(19) United States
(12) Patent Application Publication
(10) Pub. No.: US 2025/0205134 A1
(43) Pub. Date: Jun. 26, 2025

(54) OILY STICK-SHAPED COSMETIC

(71) Applicant: TOKIWA CORPORATION, Gifu (JP)

(72) Inventor: Nana KAWAGUCHI, Saitama (JP)

(73) Assignee: TOKIWA CORPORATION, Gifu (JP)

(21) Appl. No.: 18/982,360

(22) Filed: Dec. 16, 2024

(30) Foreign Application Priority Data
Dec. 25, 2023 (JP) 2023-218062

(51) Int. Cl. A61K 8/58; A61K 8/02; A61K 8/25; A61K 8/92; A61Q 1/10

(52) U.S. Cl. CPC A61K 8/585; A61K 8/0229; A61K 8/025; A61K 8/25; A61K 8/922; A61Q 1/10; A61K 2800/412; A61K 2800/872

(57) ABSTRACT

The oily stick-shaped cosmetic includes (A) a solid oil, (B) a spherical powder having an average particle diameter of 1 to 50 μm, (C) a volatile component, (D) a coating agent, and (E) a coloring material, and contains (A1) rice bran wax as the (A) component.

### TECHNICAL FIELD

The present invention relates to an oily stick-shaped cosmetic.

### BACKGROUND

An eyeliner pencil, a concealer pencil, or the like having a pencil type shape or a pencil-type that uses a retractable lead in a place where an application part is thin or narrow, such as an edge of an eyelid or a place where it is desired to hide an irregularity of a skin. As the lead material, an oily stick-shaped cosmetic composed of a solid oil such as wax and a powder component such as a coloring material is generally used, but the composition of the cosmetic is also improved according to the required function, use feeling, finish, and the like.

For example, in an eyeliner pencil, in addition to characteristics related to functions such as moldability that can retain a shape when the diameter is reduced, color development, clarity of a drawn line, and secondary adhesion resistance that can suppress secondary adhesion of a cosmetic due to blinking, a sense of use that can give feeling of smoothness and softness (creamy feeling) when applied is also required. As for finish, texture is diversified such as glossy finish, matte finish, and pearlescent finish, and in recent years, finish with matte finish tends to be preferred.

As a method for imparting a finish with a matte finish, for example, a technique of blending a silicone elastomer has been proposed in Japanese Unexamined Patent Publication No. 2000-281532 and Japanese Unexamined Patent Publication No. 2019-014670, a technique of blending a clay mineral has been proposed in Japanese Unexamined Patent Publication No. 2020-105142, and a technique of increasing the content of wax has been proposed in Japanese Unexamined Patent Publication No. 2023-62540.

### SUMMARY

However, in the technique of blending a silicone elastomer, since the silicone elastomer is mixed with a non-volatile oil, the coating film is difficult to be uniform, and secondary adhesion resistance is not sufficient. In the technique of blending a clay mineral, a dry feeling is given due to high oil absorbency, or slippage at the time of application is deteriorated, so that a creamy feeling tends to be insufficient. In the technique of increasing the content of the wax, the coating property and the creamy feeling are insufficient due to the hardness of the cosmetic, the contour of the drawn line tends to be unclear, and it tends to be difficult to achieve both color development and moldability in a thin lead shape. In addition, when the cosmetic is simply softened, a forming defect is likely to occur, the cosmetic is likely to be distorted during application (is difficult to apply the cosmetic uniformly), and the contour of the drawn line is likely to be distorted.

The present invention has been made in view of the above circumstances, and an object of the present invention is to provide an oily stick-shaped cosmetic which has sufficient moldability and can satisfy all of creamy feel when used, resistance to distort during application, and sufficient matte finish, color development, clarity of contour, and secondary adhesion resistance of a drawn line.

In order to solve the above problem, the following invention is provided.

[1] An oily stick-shaped cosmetic comprising: (A) a solid oil; (B) a spherical powder having an average particle diameter of 1 to 50 μm; (C) a volatile component; (D) a coating agent; and (E) a coloring material, and containing (A1) rice bran wax as the (A) component.
[2] The oily stick-shaped cosmetic according to [1], wherein the content of the (A) component is 3 to 25% by mass based on the total amount of the oily stick-shaped cosmetic.
[3] The oily stick-shaped cosmetic according to [1] or [2], wherein a content of the (A1) component is 0.1 to 10% by mass based on the total amount of the oily stick-shaped cosmetic.
[4] The oily stick-shaped cosmetic according to any one of [1] to [3], wherein a content of the (A1) component is 1 to 70% by mass based on the total amount of the (A) component.
[5] The oily stick-shaped cosmetic according to any one of [1] to [4], further including (A2) a hydrocarbon-based wax as the (A) component, wherein a mass ratio [(A1)/(A2)] between the (A1) component and (A2) the hydrocarbon-based wax is 1/30 to 2/1.
[6] The oily stick-shaped cosmetic according to any one of [1] to [5], wherein the (B) component is spherical silica including a metal oxide.
[7] The oily stick-shaped cosmetic according to any one of [1] to [6], wherein the (D) component is trimethylsiloxysilicate.
[8] The oily stick-shaped cosmetic according to any one of [1] to [7], wherein the content of the (C) component is 10 to 40% by mass based on the total amount of the oily stick-shaped cosmetic.
[9] The oily stick-shaped cosmetic according to any one of [1] to [8], wherein a total content of a pasty oil and a liquid oil contained in the oily stick-shaped cosmetic is 0.1 to 20% by mass based on the total amount of the oily stick-shaped cosmetic.

The oily stick-shaped cosmetic according to any one of [1] to [9], wherein a ratio of a content of the (A) component to a total content of a pasty oil and a liquid oil contained in the oily stick-shaped cosmetic is 0.1 to 30.

An oily stick-shaped cosmetic including: (A) a solid oil; (B) a spherical powder having an average particle diameter of 1 to 50 μm; (C) a volatile component; (D) a coating agent; and (E) a coloring material, and containing (A1) rice bran wax as the (A) component, wherein the (B) component is spherical silica including a metal oxide, and the (D) component is trimethylsiloxysilicate.

The oily stick-shaped cosmetic according to [11], further including (A2) a hydrocarbon-based wax as the (A) component, wherein a mass ratio [(A1)/(A2)] between the (Al) component and a hydrocarbon contained in the (A2) hydrocarbon-based wax is 1/30 to 2/1.

According to the oily stick-shaped cosmetic described in the above [1], by having the above composition, it is possible to satisfy all of sufficient moldability, creamy feel when used, resistance to distort during application, and sufficient matte finish, color development, clarity of contour, and secondary adhesion resistance of a drawn line. The present inventors infer the reason why such an effect is exhibited as follows. It is considered that, by combining and blending rice bran wax and a spherical powder having a specific size, it is possible to impart hardness capable of sufficiently retaining the shape and characteristics capable of applying the cosmetic without distorting the contour of the drawn line due to physical properties which are likely to collapse when stretched, to a cosmetic containing a solid oil such as wax which provides a matte finish and the (C) component. As a result, the present inventors infer that a drawn line can be formed while maintaining a film thickness uniform and sufficient in color development even with a weak force, and secondary adhesion resistance by the (D) component is also sufficiently obtained.

According to the present invention, it is possible to provide an oily stick-shaped cosmetic which has sufficient moldability and can satisfy all of creamy feel when used, resistance to distort during application, and sufficient matte finish, color development, clarity of contour, and secondary adhesion resistance of a drawn line.

### DETAILED DESCRIPTION

### [Oily Stick-Shaped Cosmetic]

The oily stick-shaped cosmetic of the present embodiment includes (A) a solid oil (sometimes referred to as (A) component), (B) a spherical powder having an average particle diameter of 1 to 50 μm (sometimes referred to as (B) component), (C) a volatile component (sometimes referred to as (C) component), (D) a coating agent (sometimes referred to as (D) component), and (E) a coloring material (sometimes referred to as (E) component).

### (Solid Oil)

Examples of the solid oil include waxes such as rice bran wax, beeswax, carnauba wax, candelilla wax, sunflower seed wax, spermaceti wax, and lanolin; solid oils and fats such as hydrogenated castor oil, hydrogenated jojoba oil, palm oil, and Japan wax; hydrocarbons such as paraffin wax, microcrystalline wax, candelilla wax hydrocarbon, Fischer-Tropsch wax, and polyethylene; esters such as glyceryl tribehenate and cholesterol fatty acid ester; higher alcohols such as stearyl alcohol and behenyl alcohol, silicones such as acryl-modified silicone, and sugar fatty acid esters such as dextrin palmitate and inulin stearate.

In the present specification, the solid oil refers to an oil that does not exhibit fluidity at 40° C. The solid oil may have a melting point of 40° C. or higher. The melting point of the solid oil is measured by the following method.

About 5 mg of the sample is weighed and placed in an aluminum sample pan. An aluminum cover is attached to the pan, and the pan is placed in a differential scanning calorimeter “DSC 7020” (manufactured by Hitachi High-Tech Science Corporation, product name). Using an electric cooling unit “Polyscience” (manufactured by Hitachi High-Tech Science Corporation, product name), a sample and a reference sample are held at −10° C. for 1 minute at a nitrogen gas flow rate of 30 to 35 mL/min, then the temperature is raised from 0° C. to 120° C. at a temperature rising rate of 10° C./min, the temperature is lowered from 120° C. to −10° C. at a temperature falling condition of −10° C./min, the sample is held at −10° C. for 5 minutes, and then the temperature is raised again from −10° C. to 120° C. at a temperature rising rate of 10° C./min, thereby obtaining a melting endothermic curve. The peak temperature of the melting endothermic curve at the second temperature rise at this time is taken as the melting point.

The (A) components can be used singly or in combination of two or more kinds thereof.

The content of the (A) component may be 3 to 25% by mass, 5 to 20% by mass, or 7 to 18% by mass based on the total amount of the oily stick-shaped cosmetic from the viewpoints of moldability, a creamy feel when used, and resistance to distort during application.

The oily stick-shaped cosmetic of the present embodiment contains, as the (A) component, (A1) rice bran wax (also referred to as the (A1) component in some cases) from the viewpoint of forming a drawn line having sufficient moldability, creamy feel when used, resistance to distort during application, and sufficient matte finish, color development, clarity of contour, and secondary adhesion resistance.

As rice bran wax, those containing an ester of an alcohol with C22 to C36 and a fatty acid with C16 to C34 can be used.

As rice bran wax, commercially available products such as “NC-1720” (manufactured by CERARICA NODA Co., Ltd., product name), “ORYZA SATIVA (RICE) BRAN WAX SS-2” (manufactured by Boso oil and fat Co., Ltd., product name), and “refined rice wax S-100” (Product name, manufactured by YOKOZEKI OIL & FAT INDUSTRIES Co., LTD.) can be used.

The saponification value of rice bran wax may be 70 to 95 or 75 to 95. The melting point of rice bran wax may be 70 to 90° C. or 75 to 85° C.

The content of the (A1) component may be 0.1 to 10% by mass, 0.3 to 8% by mass, or 0.5 to 7% by mass based on the total amount of the oily stick-shaped cosmetic from the viewpoint of satisfying the creamy feel when used, the sufficient moldability, the resistance to distort during application, and the clarity of the contour of the drawn line at the high level.

In addition, the content of the (A1) component may be 1 to 70% by mass, 5 to 60% by mass, or 7 to 40% by mass, based on the total amount of the (A) component, from the viewpoint of moldability and creamy feel when used.

The oily stick-shaped cosmetic of the present embodiment may contain, as the (A) component, the (A1) component and a hydrocarbon-based wax (also referred to as a (A2) component) from the viewpoint of further improving moldability, color development, and clarity of contour. In the present specification, the hydrocarbon-based wax refers to a solid oil containing 30% by mass or more of hydrocarbon. The (A2) component can be used singly or in combination of two or more kinds thereof.

As the (A2) component, from the viewpoint of moldability, the above-described hydrocarbons, candelilla wax, and the like can be used. The (A2) component may contain polyethylene from the viewpoint of moldability. The (A2) component can be used singly or in combination of two or more kinds thereof.

When the oily stick-shaped cosmetic of the present embodiment further contains the (A2) component as the (A) component, the mass ratio of the (A1) component to the hydrocarbon contained in the (A2) component [(A1)/(A2)] may be 1/30 to 2/1, 1/25 to 4/3, or 1/12 to 1/1 from the viewpoint of further improving the moldability, the color development, and the clarity of the contour. In addition, from the viewpoint of a creamy feel when used and no distortion, the total content of hydrocarbons contained in the (A1) component and the (A2) component may be 3 to 25% by mass, 5 to 20% by mass, 6 to 19% by mass, or 8 to 18% by mass based on the total amount of the oily stick-shaped cosmetic.

### (Spherical Powder Having Average Particle Diameter of 1 to 50 Mm)

As the (B) component, a spherical composite powder containing spherical inorganic particles, spherical organic particles, and a metal oxide can be used. The spherical composite powder containing the metal oxide may be spherical inorganic particles including the metal oxide.

In the present specification, the average particle diameter of the spherical powder is a value of a volume average particle diameter measured by a laser diffraction scattering particle diameter distribution analyzer (LA-960 manufactured by HORIBA, Ltd.) in a dry powder state. The volume average particle diameter is a volume-based average particle diameter, and is defined as a 50% median diameter. The average particle diameter of the spherical powder may be determined with reference to the catalog value of the raw material.

Examples of the spherical inorganic particles include silica, calcium carbonate, and magnesium carbonate. These may be porous. As the spherical inorganic particles, commercially available products such as “SUNSIL 130 L” (manufactured by Sunjin Chemical Co., Ltd., product name, average particle diameter: 7 μm), “silica microbead P-1500” (manufactured by JGC Catalysts and Chemicals Ltd., product name, average particle diameter: 11 μm), and “spherical calcium carbonate Calmaru SCS-M5” (manufactured by Sakai Chemical Industry Co., Ltd., product name, average particle diameter: 5 μm) may be used.

Examples of the spherical organic particles include methyl methacrylate cross polymer, nylon spherical powder, crosslinked silicone, silk powder, and urethane powder. As the spherical organic particles, commercially available products such as “Matsumoto Microsphere M503B” (manufactured by Matsumoto Yushi-Seiyaku Co., Ltd., product name, average particle diameter: 20 μm), “Nylon-12” (manufactured by Toray Industries, Inc., product name, average particle diameter: 5 μm), and “KSP-101” (manufactured by Shin-Etsu Chemical Co., Ltd., product name, average particle diameter: 5 μm) may be used.

Examples of the metal oxide constituting the spherical composite powder containing the metal oxide include titanium oxide, zinc oxide, and iron oxide.

The average particle diameter of the metal oxide may be 1 to 1000 nm, 3 to 500 nm, 5 to 100 nm, or 8 to 50 nm.

Examples of the matrix of the composite powder include silica, titanium oxide, zinc oxide, and iron oxide.

The spherical composite powder containing the metal oxide may be spherical inorganic particles (For example, the parent body described above) including the metal oxide. As such a composite powder, a commercially available product can be used. Examples thereof include “SUNSIL TIN 40” (manufactured by Sunjin Chemical Co., Ltd., average particle diameter: 5 μm), “SUNSIL TIN 50” (manufactured by Sunjin Chemical Co., Ltd., product name, average particle diameter: 5 μm), “SH219” (manufactured by Sunjin Chemical Co., Ltd., product name, average particle diameter: 5 μm), “SH219AS” (manufactured by Sunjin Chemical Co., Ltd., product name, average particle diameter: 5 μm), “PC ball WT-35H” (manufactured by SUZUKIYUSHI INDUSTRIAL CORPORATION, product name, average particle diameter: 3 μm), “PULCEAR SIZ-30” (manufactured by SUZUKIYUSHI INDUSTRIAL CORPORATION, product name, volume average particle diameter: 4 μm), and “i-NAFLECS IWS22S13” (manufactured by Nippon Sheet

Glass Corporation, volume average particle diameter: 4 μm). The oily stick-shaped cosmetic of the present embodiment may contain spherical silica including titanium oxide as the (B) component from the viewpoint of further improving the creamy feel when used, the color development of the drawn line, and the clarity of the contour.

The (B) component can be used singly or in combination of two or more kinds thereof.

The content of the (B) component may be 0.5 to 35% by mass, 1 to 30% by mass, or 3 to 20% by mass based on the total amount of the oily stick-shaped cosmetic from the viewpoint of satisfying the creamy feel when used, the sufficient moldability, the resistance to distort during application, and the clarity of the contour of the drawn line at the high level.

### (Volatile Component)

Examples of the (C) component include volatile silicone oil and volatile hydrocarbon oil.

Examples of the volatile silicone oil include low-molecular-weight linear silicones such as dimethylpolysiloxane, cyclic silicones such as octamethylcyclotetrasiloxane and decamethylcyclopentasiloxane, and branched silicones such as methyltrimethicone.

Examples of the volatile hydrocarbon oil include isododecane, undecane, tridecane, and light liquid isoparaffin.

The (C) component may have a boiling point of 250° C. or lower, 210° C. or lower, or 200° C. or lower at 1 atm (101.325 kPa).

The (C) component can be used singly or in combination of two or more kinds thereof.

The content of the (C) component may be 10 to 40% by mass, 15 to 30% by mass, or 17 to 27% by mass based on the total amount of the oily stick-shaped cosmetic from the viewpoint of a creamy feel when used, resistance to distort during application, and secondary adhesion resistance.

### (Coating Agent)

Examples of the (D) component include silicone coating agents such as silicone resins such as trimethylsiloxysilicate and polyalkylsilsesquioxane, acrylic/silicone resins such as (acrylate/stearyl acrylate/dimethicone methacrylate) copolymer and (acrylate/dimethicone) copolymer, and candelilla resin. From the viewpoint of balancing color development, secondary adhesion resistance, and creamy feel when used, the (D) component may be trimethylsiloxysilicate.

The (D) component can be used singly or in combination of two or more kinds thereof.

The content of the (D) component may be 3 to 35% by mass or 5 to 30% by mass based on the total amount of the oily stick-shaped cosmetic from the viewpoint of color development and secondary adhesion resistance, and may be 10 to 28% by mass from the viewpoint of creamy feel when used and moldability.

### (Coloring Material)

Examples of the (E) component include a colored inorganic pigment, a colored organic pigment, and a pearl pigment.

Examples of the colored inorganic pigment include red iron oxide, yellow iron oxide, black iron oxide, cobalt oxide, ultramarine, Prussian blue, titanium oxide, and zinc oxide. Examples of the pearl pigment include one obtained by coating a surface of mica, synthetic phlogopite, glass, silica, alumina, talc, or the like with a coloring agent such as titanium oxide, iron oxide, silicon oxide, Prussian blue, chromium oxide, tin oxide, chromium hydroxide, gold, silver, carmine, or an organic pigment, titanium mica, a fish scale foil, bismuth oxychloride, and aluminum flakes. Examples of the colored organic pigment include red 228, red 226, blue 404, red 202, red 201, yellow 4, aluminum lake, and blue 1), and natural dyes (Carmine, safflower, etc.).

The (E) component can be used singly or in combination of two or more kinds thereof.

When the oily stick-shaped cosmetic of the present embodiment contains the pearl pigment as the (E) component, since the coating film has a matte finish, a drawn line in which the pearlescent finish (glitter) by the pearl pigment is further enhanced can be formed.

The content of the (E) component may be 5 to 50% by mass, 8 to 40% by mass, or 10 to 35% by mass based on the total amount of the oily stick-shaped cosmetic from the viewpoint of moldability, color development, and creamy feel when used.

### (Other Components)

The oily stick-shaped cosmetic of the present embodiment can be used without particular limitation as a component other than the above components (A) to (E), for example, an oily component (other oily components) other than the (A) component, the (C) component, and the (D) component, and a powder component (other powder components) other than the (B) component and the (E) component as long as the component is usually used in cosmetics. The other component can be used singly or in combination of two or more kinds thereof.

As the other oily component, an oil agent other than the (A) component may be used, and for example, a pasty oil or a liquid oil can be used. Examples of the pasty oil include vaseline, dipentaerythrityl hexa (hydroxystearic acid/stearic acid/rosin acid), dipentaerythrityl tetra (hydroxystearic acid/isostearic acid), dipentaerythrityl pentaisostearate, dipentaerythrityl hexahydroxystearate, tri (caprylic acid/capric acid/myristic acid/stearic acid) glyceryl, hydrogenated castor oil isostearate, phytosteryl oleate, sucrose hexa (oleic acid/palmitic acid/stearic acid), phytosteryl macadamiate, bis-diglyceryl polyacyladipate-2, phytosteryl/isostearyl/cetyl/stearyl/behenyl dimer dilinoleate.

Examples of the liquid oil include ester oils such as sorbitan sesquiisostearate, sorbitan isostearate, sorbitan sesquioleate, cetyl ethylhexanoate, ethylhexyl palmitate, triethylhexanoin, isotridecyl isononanoate, isostearyl isostearate, neopentyl glycol diethylhexanoate, tri (capryl/capric acid) glyceryl, neopentyl glycol dicaprate, propanediol diisostearate, trimethylolpropane triethylhexanoate, octyldodecyl myristate, octyldodecyl stearoyloxystearate, diisostearyl malate, polyglyceryl triisostearate, dipentaerythrityl pentaisostearate, and trimethylolpropane triisostearate; silicone oils such as non-volatile dimethicone and methylphenylpolysiloxane; hydrocarbon oils such as liquid paraffin, squalane, olefin oligomer, liquid paraffin, squalane, and hydrogenated polyisobutene; plant oils such as sunflower seed oil, jojoba seed oil, olive oil, castor oil; higher alcohols such as isostearyl alcohol, octyldodecanol, oleyl alcohol.

In the oily stick-shaped cosmetic of the present embodiment, the total content of the pasty oil and the liquid oil may be 0.1 to 20% by mass, 0.5 to 15% by mass, or 1 to 10% by mass based on the total amount of the oily stick-shaped cosmetic. In the present specification, the liquid oil means an oily component having a melting point of 25° C. or less and a viscosity of 1 million mPa's or less at 25° C.

In the oily stick-shaped cosmetic of the present embodiment, the ratio of the content of the (A) component to the total content of the pasty oil and the liquid oil may be 0.1 to 30, 0.2 to 20, or 0.3 to 16 from the viewpoint of further improving the creamy feel when used, and the matte finish, color development, clarity of contour, and secondary adhesion resistance of the drawn line.

The other powder component can be used without particular limitation as long as it is usually a powder used for cosmetics, and examples thereof include a body powder other than the (B) component. The shape of the powder is not particularly limited, and the powder may have a spherical shape, a plate shape, a needle shape, or the like, or a particle structure such as a haze shape, fine particles, a pigment particle diameter, porosity, or non-porosity.

Specific examples thereof include extender pigments such as mica, synthetic mica, sericite, talc, kaolin, silicon carbide, barium sulfate, bentonite, smectite, aluminum oxide, plate-shaped silica, magnesium oxide, zirconium oxide, chromium oxide, aluminum hydroxide, and magnesium hydroxide; ultraviolet scattering agents such as fine particles of titanium oxide and fine particles of zinc oxide; organic powders such as acrylonitrile-methacrylic acid copolymer powder, vinylidene chloride-methacrylic acid copolymer, polyethylene powder, polystyrene powder, polymethylsilsesquioxane powder, wool powder, cellulose powder, and N-acyl lysine powder; and metal soaps such as magnesium stearate, zinc myristate, aluminum stearate, and calcium stearate.

These powder components may be hydrophobically treated powder. Examples of the hydrophobic treatment include higher fatty acids, metal soaps, fats and oils, waxes, silicone compounds, fluorine compounds, surfactants, and dextrin fatty acid esters.

In the oily stick-shaped cosmetic of the present embodiment, the total content of the oily component (including the (A) component, the (C) component, and the (D) component) may be 30 to 90% by mass, 35 to 85% by mass, or 40 to 80% by mass based on the total amount of the oily stick-shaped cosmetic.

In the oily stick-shaped cosmetic of the present embodiment, the total content of the powder components (including the (B) component and the (E) component) may be 10 to 70% by mass, 15 to 65% by mass, or 20 to 60% by mass based on the total amount of the oily stick-shaped cosmetic.

In addition to the above components, the oily stick-shaped cosmetic of the present embodiment may contain components usually used in cosmetics, for example, surfactants, antiseptics, antioxidants, dyes, thickeners, pH adjusters, fragrances, ultraviolet absorbers, ultraviolet scattering agents, chelating agents, anti-inflammatory agents, moisturizers, and the like.

The oily stick-shaped cosmetic of the present embodiment can be produced, for example, by the following procedure. An oily component, other components as necessary, and the like are mixed and heated and dissolved. The powder component is mixed with the obtained solution, and the mixture is uniformly dispersed with a three-roll machine or a stirrer. Thereafter, the mixture (cosmetic base material) is molded by an extruder, mold molding, container filling, or the like, whereby an oily stick-shaped cosmetic can be obtained.

The shape of the oily stick-shaped cosmetic is not particularly limited, but the shape of the cross section orthogonal to the major axis direction of the oily stick-shaped cosmetic may be, for example, a circle, an ellipse, a triangle, a rectangle, a polygon (the number of corners is 5 or more), a drop shape, a heart shape, or the like.

When the cross-sectional shape is a circle or an ellipse, the minor axis passing through the diameter or the center of the ellipse may be 0.5 to 10 mm, 1.0 to 5.0 mm, or 1.3 to 3.0 mm.

When the cross-sectional shape is a shape other than a circle and an ellipse, the shortest part diameter passing through the center may be 0.5 to 10 mm, 1.0 to 5 mm, or 1.5 to 3 mm regardless of the shape as long as the outer edge is included in a circle having a diameter of 10 mm.

The oily stick-shaped cosmetic of the present embodiment is suitably used as an eyeliner, an eyeshadow, a concealer, a highlight, a lip liner, a tear bag makeup, and the like. The oily stick-shaped cosmetic of the present embodiment can be used for a mechanical container in which a rod-shaped cosmetic is retractable and used, or a pencil type cosmetic product incorporated in a wooden or resin shaft container or the like.

The creamy feel when used of the oily stick-shaped cosmetic of the present embodiment is effective for imparting a desired cosmetic effect with a good finish to the face (particularly, the edge of an eye) that is difficult to draw with force.

### EXAMPLES

Hereinafter, the present invention will be described in more detail with reference to Examples, but the technical scope of the present invention is not limited by these Examples.

### [Preparation of Oily Stick-Shaped Cosmetic]

### Examples 1 to 27 and Comparative Examples 1 to 3

An oily stick-shaped cosmetic (eyeliner) (in Example 18, eyeshadow) shown in Tables 1 to 5 was prepared by the following production method.

### <Production Method>

The powder component was mixed with a mixture in which the oily component was dissolved at 80 to 100° C., and the mixture was uniformly dispersed using a 3 roll to obtain a cosmetic base material. The obtained cosmetic base material was filled in a mold and molded into a cylindrical shape having a diameter of 2 mm. In addition, Example 18 was molded into a cylindrical shape having a diameter of 5 mm.

### [Evaluation of Oily Stick-Shaped Cosmetic]

The obtained oily stick-shaped cosmetic was evaluated for each item based on the following evaluation method. The results are shown in Tables 1 to 5.

<Evaluation of Feel when Used and Finish>

Five expert panels on cosmetics evaluation evaluated the products from the viewpoints of a creamy feel when used, clarity of the contour of the drawn line, no distortion, matte finish, and good color development (concealing property) using a retractable pencil container in which the cosmetics of Examples and Comparative Examples were accommodated as a lead material, the cosmetic was applied to the edge of an eye. In each evaluation, five-grade evaluation was performed according to the following evaluation criteria, a score was given to each sample, and an average score of the scores of all the panels was determined according to the following determination criteria.

### [Score: Evaluation Criteria]

5 Points: very good
4 Points: good
3 Points: normal
2 Points: slightly poor
1 Point: poor

### [Determination Criteria (Average Score)]

⊙: 4 or more
◯: 3 or more and less than 4
Δ: 2 or more and less than 3
X: less than 2

### <Secondary Adhesion Resistance>

The cosmetic was applied to a Bio-skin plate (manufactured by Beaulax Co., Ltd) in a size of 1 cm×3 cm, and after 5 minutes, the plate was rubbed back and forth three times with a finger, and visually evaluated in four stages according to the following evaluation criteria.

### [Determination Criteria]

⊙: The cosmetic did not adhere to the finger, and the coating film was not changed.
∘: The cosmetic was slightly transferred to the finger, but the coating film was not changed.
Δ: The cosmetic is slightly transferred to the finger, and the coating film becomes thin.
X: The cosmetic was transferred to the finger, and almost no coating film remained.

### <Moldability>

The cosmetic base material was filled in a mold having a rod-like shape, cooled and solidified, and then the state of the taken-out molded product was visually evaluated in four stages according to the following evaluation criteria.

### [Determination Criteria]

⊙: The molded product is molded in the shape of a mold.
Δ: The molded product is slightly bent.
X: The molded product is bent, or the cosmetic base material is not solidified or separated.

TABLE 1

| | | | Example | Example | Example | Example | Example | Example | Example |
|---|---|---|---|---|---|---|---|---|---|
| | | | 1 | 2 | 3 | 4 | 5 | 6 | 7 |
| Oily | (A) | (A1) Rice bran wax | 0.5 | 1 | 3 | 5 | 8 | 8 | 5.7 |
| component | Component | Polyethylene | 9 | 9 | 9 | 7 | 4 | 6.2 | 9 |
| | | Candelilla wax | 5.2 | 4.7 | 2.7 | 2.7 | 2.7 | 0.5 | — |
| | (C) | Volatile dimethicone | 26 | 26 | 26 | 26 | 26 | 26 | 26 |
| | Component | | | | | | | | |
| | (D) | Trimethylsiloxysilicate | 24.5 | 24.5 | 24.5 | 24.5 | 24.5 | 24.5 | 24.5 |
| | Component | | | | | | | | |
| | Others | Sorbitan | 1.5 | 1.5 | 1.5 | 1.5 | 1.5 | 1.5 | 1.5 |
| | | sesquiisostearate | | | | | | | |
| | | Nonvolatile | 1.3 | 1.3 | 1.3 | 1.3 | 1.3 | 1.3 | 1.3 |
| | | dimethicone | | | | | | | |
| | | Diisostearyl malate | 2 | 2 | 2 | 2 | 2 | 2 | 2 |
| Powder | (B) | Spherical powder B-1 | 4 | 4 | 4 | 4 | 4 | 4 | 4 |
| component | Component | | | | | | | | |
| | (E) | Titanium oxide | 3.2 | 3.2 | 3.2 | 3.2 | 3.2 | 3.2 | 3.2 |
| | Component | Black iron oxide | 2 | 2 | 2 | 2 | 2 | 2 | 2 |
| | | Red iron oxide | 19.8 | 19.8 | 19.8 | 19.8 | 19.8 | 19.8 | 19.8 |
| | Others | Hydrophobic synthetic | 1.0 | 1.0 | 1.0 | 1.0 | 1.0 | 1.0 | 1.0 |
| | | mica | | | | | | | |

| | | | | | | | | |
|---|---|---|---|---|---|---|---|---|
| Evaluation | Creamy feel when used | ◯ | ⊙ | ⊙ | ⊙ | ◯ | ⊙ | ⊙ |
| | Clarity of contour of drawn line | ⊙ | ⊙ | ⊙ | ⊙ | ⊙ | ⊙ | ⊙ |
| | No distortion | ⊙ | ⊙ | ⊙ | ⊙ | ⊙ | ⊙ | ⊙ |
| | Matte finish | ⊙ | ⊙ | ⊙ | ⊙ | ⊙ | ⊙ | ⊙ |
| | Good color development | ◯ | ⊙ | ⊙ | ◯ | ◯ | ◯ | ⊙ |
| | (concealing property) | | | | | | | |
| | Secondary adhesion resistance | ⊙ | ⊙ | ⊙ | ⊙ | ⊙ | ⊙ | ⊙ |
| | Moldability | ⊙ | ⊙ | ⊙ | ⊙ | Δ | ⊙ | ⊙ |

TABLE 2

| | | | Example | Example | Example | Example | Example | Example | Example |
|---|---|---|---|---|---|---|---|---|---|
| | | | 8 | 9 | 10 | 11 | 12 | 13 | 14 |
| Oily | (A) | (A1) Rice bran wax | 3 | 3 | 3 | 3 | 3 | 3 | 3 |
| component | Component | Polyethylene | 9 | 9 | 9 | 9 | 9 | 9 | 9 |
| | | Candelilla wax | 2.7 | 2.7 | 2.7 | 2.7 | 2.7 | 2.7 | 2.7 |
| | (C) | Volatile dimethicone | 26 | 26 | 26 | 26 | 26 | 26 | 26 |
| | Component | | | | | | | | |
| | (D) | Trimethylsiloxysilicate | 24.5 | 24.5 | 24.5 | 24.5 | 24.5 | 24.5 | 24.5 |
| | Component | | | | | | | | |
| | Others | Sorbitan | 1.5 | 1.5 | 1.5 | 1.5 | 1.5 | 1.5 | 1.5 |
| | | sesquiisostearate | | | | | | | |
| | | Nonvolatile | 1.3 | 1.3 | 1.3 | 1.3 | 1.3 | 1.3 | 1.3 |
| | | dimethicone | | | | | | | |
| | | Diisostearyl malate | 2 | 2 | 2 | 2 | 2 | 2 | 2 |
| Powder | (B) | Spherical powder B-1 | 1 | 10 | 15 | 20 | — | — | — |
| component | Component | Spherical powder B-2 | — | — | — | — | 4 | — | — |
| | | Spherical powder B-3 | — | — | — | — | — | 4 | — |
| | | Spherical powder B-4 | — | — | — | — | — | — | 4 |
| | (E) | Titanium oxide | 3.2 | 2.5 | 1.1 | 0.7 | 3.2 | 3.2 | 3.2 |
| | Component | Black iron oxide | 2.3 | 1.6 | 1.3 | 0.9 | 2 | 2 | 2 |
| | | Red iron oxide | 15.2 | 15.8 | 12.5 | 8.3 | 19.8 | 19.8 | 19.8 |
| | Others | Hydrophobic synthetic | 1.0 | 1.0 | 1.0 | 1.0 | 1.0 | 1.0 | 1.0 |
| | | mica | | | | | | | |

| | | | | | | | | |
|---|---|---|---|---|---|---|---|---|
| Evaluation | Creamy feel when used | ◯ | ⊙ | ⊙ | ⊙ | ⊙ | ◯ | ⊙ |
| | Clarity of contour of drawn line | Δ | ⊙ | ◯ | ◯ | ⊙ | ⊙ | Δ |
| | No distortion | ⊙ | ⊙ | ◯ | ◯ | ⊙ | ⊙ | ⊙ |
| | Matte finish | ◯ | ⊙ | ⊙ | ⊙ | ⊙ | ⊙ | ⊙ |
| | Good color development | Δ | ⊙ | ⊙ | ⊙ | ⊙ | ◯ | ⊙ |
| | (concealing property) | | | | | | | |
| | Secondary adhesion resistance | ⊙ | ⊙ | ⊙ | ⊙ | ⊙ | ⊙ | ⊙ |
| | Moldability | ⊙ | ⊙ | ⊙ | ⊙ | ⊙ | ⊙ | ⊙ |

TABLE 3

| | | | Example | Example | Example | Example | Example | Example | Example |
|---|---|---|---|---|---|---|---|---|---|
| | | | 15 | 16 | 17 | 18 | 19 | 20 | 21 |
| Oily | (A) | (A1) Rice bran wax | 4.1 | 2 | 1.2 | 1.2 | 3 | 3 | 3 |
| component | Component | Polyethylene | 12.2 | 6 | 3.7 | 3.7 | 9 | 9 | 9 |
| | | Candelilla wax | 3.7 | 1.8 | 1.1 | 1.1 | 2.7 | 2.7 | 2.7 |
| | (C) | Volatile dimethicone | 26 | 26 | 26 | 26 | 26 | 21 | 16 |
| | Component | | | | | | | | |
| | (D) | Trimethylsiloxysilicate | 24.5 | 24.5 | 24.5 | 24.5 | 24.5 | 24.5 | 24.5 |
| | Component | | | | | | | | |
| | Others | Sorbitan | 1.5 | 1.5 | 1.5 | 1.5 | 0.4 | 3 | 4.5 |
| | | sesquiisostearate | | | | | | | |
| | | Nonvolatile | 1.3 | 1.3 | 1.3 | 1.3 | 0.6 | 3 | 4.5 |
| | | dimethicone | | | | | | | |
| | | Diisostearyl malate | 2 | 2 | 2 | 2 | 1 | 4 | 6 |
| Powder | (B) | Spherical powder B-1 | 4 | 4 | 4 | 4 | 4 | 4 | 4 |
| component | Component | | | | | | | | |
| | (E) | Titanium oxide | 2.5 | 3.2 | 3.2 | 3.2 | 3.2 | 3.2 | 3.2 |
| | Component | Black iron oxide | 1.6 | 2 | 2 | 2 | 2 | 2 | 2 |
| | | Red iron oxide | 15.4 | 19.8 | 19.8 | 19.8 | 19.8 | 19.8 | 19.8 |
| | Others | Hydrophobic synthetic | 1.2 | 5.9 | 9.7 | 9.7 | 3.8 | 0.8 | 0.8 |
| | | mica | | | | | | | |

| | | | | | | | | |
|---|---|---|---|---|---|---|---|---|
| Evaluation | Creamy feel when used | ◯ | ⊙ | ⊙ | ⊙ | ⊙ | ⊙ | ⊙ |
| | Clarity of contour of drawn line | ⊙ | ⊙ | ⊙ | ⊙ | ⊙ | ⊙ | ⊙ |
| | No distortion | ⊙ | ⊙ | Δ | ⊙ | ⊙ | ⊙ | ⊙ |
| | Matte finish | ⊙ | ⊙ | ⊙ | ⊙ | ⊙ | ⊙ | Δ |
| | Good color development | ◯ | ⊙ | ⊙ | ⊙ | ⊙ | ⊙ | ◯ |
| | (concealing property) | | | | | | | |
| | Secondary adhesion resistance | ⊙ | ⊙ | ⊙ | ⊙ | ⊙ | ⊙ | Δ |
| | Moldability | ⊙ | ⊙ | ⊙ | ⊙ | ⊙ | ⊙ | ⊙ |

TABLE 4

| | | | Example | Example | Example | Example | Example | Example |
|---|---|---|---|---|---|---|---|---|
| | | | 22 | 23 | 24 | 25 | 26 | 27 |
| Oily | (A) | (A1) Rice bran wax | 3 | 3 | 3 | 3 | 3 | 3 |
| component | Component | Polyethylene | 9 | 9 | 9 | 9 | — | — |
| | | Candelilla wax | — | — | — | — | 2.7 | 2.7 |
| | | Carnauba wax | 2.7 | — | — | — | — | — |
| | | Beeswax | — | 2.7 | — | — | — | — |
| | | Microcrystalline wax | — | — | 2.7 | — | — | — |
| | | Sunflower seed wax | — | — | — | 2.7 | — | — |
| | | Candelilla wax | — | — | — | — | 9 | — |
| | | hydrocarbon | | | | | | |
| | | Paraffin wax | — | — | — | — | — | 9 |
| | (C) | Volatile dimethicone | 26 | 26 | 26 | 26 | 26 | 26 |
| | Component | | | | | | | |
| | (D) | Trimethylsiloxysilicate | 24.5 | 24.5 | 24.5 | 24.5 | 24.5 | 24.5 |
| | Component | | | | | | | |
| | Others | Sorbitan | 1.5 | 1.5 | 1.5 | 1.5 | 1.5 | 1.5 |
| | | sesquiisostearate | | | | | | |
| | | Nonvolatile | 1.3 | 1.3 | 1.3 | 1.3 | 1.3 | 1.3 |
| | | dimethicone | | | | | | |
| | | Diisostearyl malate | 2 | 2 | 2 | 2 | 2 | 2 |
| Powder | (B) | Spherical powder B-1 | 4 | 4 | 4 | 4 | 4 | 4 |
| component | Component | | | | | | | |
| | (E) | Titanium oxide | 3.2 | 3.2 | 3.2 | 3.2 | 3.2 | 3.2 |
| | Component | Black iron oxide | 2 | 2 | 2 | 2 | 2 | 2 |
| | | Red iron oxide | 19.8 | 19.8 | 19.8 | 19.8 | 19.8 | 19.8 |
| | Others | Hydrophobic synthetic | 1.0 | 1.0 | 1.0 | 1.0 | 1.0 | 1.0 |
| | | mica | | | | | | |

| | | | | | | | |
|---|---|---|---|---|---|---|---|
| Evaluation | Creamy feel when used | ◯ | ⊙ | ⊙ | ⊙ | ⊙ | ⊙ |
| | Clarity of contour of drawn line | ⊙ | ⊙ | ⊙ | ⊙ | ◯ | ⊙ |
| | No distortion | ⊙ | ⊙ | ⊙ | ⊙ | ⊙ | ⊙ |
| | Matte finish | ⊙ | ⊙ | ⊙ | ⊙ | ⊙ | ◯ |
| | Good color development | ◯ | ⊙ | ⊙ | ⊙ | ◯ | ⊙ |
| | (concealing property) | | | | | | |
| | Secondary adhesion resistance | ⊙ | ⊙ | ⊙ | ⊙ | ⊙ | ⊙ |
| | Moldability | ⊙ | ⊙ | ⊙ | ⊙ | ⊙ | ⊙ |

TABLE 5

| | | | Comparative | Comparative | Comparative |
|---|---|---|---|---|---|
| | | | Example 1 | Example 2 | Example 3 |
| Oily | (A) | (A1) Rice bran wax | — | 3 | 3 |
| component | Component | Polyethylene | 9 | 9 | 9 |
| | | Candelilla wax | 5.7 | 2.7 | 2.7 |
| | (C) | Volatile dimethicone | 26 | 26 | 26 |
| | Component | | | | |
| | (D) | Trimethylsiloxysilicate | 24.5 | 24.5 | 24.5 |
| | Component | | | | |
| | Others | Sorbitan | 1.5 | 1.5 | 1.5 |
| | | sesquiisostearate | | | |
| | | Nonvolatile | 1.3 | 1.3 | 1.3 |
| | | dimethicone | | | |
| | | Diisostearyl malate | 2 | 2 | 2 |
| Powder | (B) | Spherical powder B-1 | 4 | — | — |
| component | Component | | | | |
| | Others | Plate-shaped powder | — | 4 | — |
| | | P-1 | | | |
| | (E) | Titanium oxide | 3.2 | 3.2 | 3.2 |
| | Component | Black iron oxide | 2 | 2 | 2 |
| | | Red iron oxide | 19.8 | 19.8 | 19.8 |
| | Others | Hydrophobic synthetic | 1.0 | 1.0 | 1.0 |
| | | mica | | | |

| | | | | |
|---|---|---|---|---|
| Evaluation | Creamy feel when used | X | X | X |
| | Clarity of contour of drawn line | Δ | X | Δ |
| | No distortion | Δ | ⊙ | ⊙ |
| | Matte finish | ⊙ | ◯ | Δ |
| | Good color development | ⊙ | X | X |
| | (concealing property) | | | |
| | Secondary adhesion resistance | ⊙ | ⊙ | ⊙ |
| | Moldability | ⊙ | ⊙ | ⊙ |

Details of the solid oils and the powder components shown in Tables 1 to 5 and the following Examples are as follows.

### (Solid Oil)

Rice bran wax: “NC-1720” (Product name, manufactured by CERARICA NODA Co., Ltd.)
Polyethylene: “PERFORMA SW-84 Synthetic Wax” (Product name, manufactured by NuCera Solutions LLC)
Candelilla wax: “purified Candelilla wax MK-4” (manufactured by YOKOZEKI OIL & FAT INDUSTRIES Co., LTD., product name, hydrocarbon content: about 40% by mass)
Candelilla wax hydrocarbon: “purified Candelilla wax MD-21” (manufactured by YOKOZEKI OIL & FAT INDUSTRIES Co., LTD., product name, hydrocarbon content: about 80% by mass)

### (Powder Component)

Spherical powder B-1: spherical silica including titanium oxide (12 nm) (Product name “SH219” manufactured by Sunjin Chemical Co., Ltd., average particle diameter: 5 μm)
Spherical powder B-2: Hydrophobic titanium oxide (12 nm)-including spherical silica (Product name “SH219AS” manufactured by Sunjin Chemical Co., Ltd., average particle diameter: 5 μm)
Spherical powder B-3: spherical silica (manufactured by Sunjin Chemical Co., Ltd., product name “SUNSIL 130L”, average particle diameter: 7 μm)
Spherical powder B-4: methyl methacrylate cross polymer (manufactured by Matsumoto Yushi-Seiyaku Co., Ltd., product name: “Matsumoto Microsphere M503B”, average particle diameter: 20 μm)
Plate-shaped powder P-1: plate-shaped silica (Product name “OVEIL DR1” manufactured by Osaka Gas Chemicals Co., Ltd., particle diameter: 11 μm)

### Example 28: Eyeliner

| | (blending ratio |
|---|---|
| (Component) | (% by mass)) |
| 1. Rice bran wax | 2.5 |
| 2. Polyethylene | 8.0 |
| 3. Candelilla wax | 2.7 |
| 4. Volatile dimethicone | 22.0 |
| 5. Trimethylsiloxysilicate | 25.0 |
| 6. Sorbitan sesquiisostearate | 1.5 |
| 7. Nonvolatile dimethicone | 1.3 |
| 8. Diisostearyl malate | 2.3 |
| 9. Spherical Powder B-1 | 2.0 |
| 10. Mica base pearl agent (particle diameter: 20 μm) | 12.0 |
| 11. Synthetic Mica | 6.68 |
| 12. Titanium oxide | 14.0 |
| 13. Antioxidant | 0.02 |

### <Production Method>

The powder component was mixed with a mixture in which the oily component was dissolved at 80 to 100° C., and the mixture was uniformly dispersed using a 3 roll to obtain a cosmetic base material. The obtained cosmetic base material was filled in a mold and molded into a cylindrical shape having a diameter of 2 mm.

### <Evaluation>

The obtained eyeliner was evaluated in the same manner as described above, and consequently it was confirmed that the eyeliner was evaluated as creamy feel when used “◯”, clarity of contour of drawn lines “⊙”, no distortion “⊙”, matte finish “⊙”, good color development (concealing property) “◯”, secondary adhesion resistance “⊙”, and moldability “⊙”. The above eyeliner was evaluated for pearlescent finish in addition to matte finish, and the result was evaluated as “⊙”.

### Example 29: Eyeliner

| | blending ratio |
|---|---|
| (Component) | (% by mass)) |
| 1. Rice bran wax | 2.3 |
| 2. Polyethylene | 8.0 |
| 3. Candelilla wax | 1.8 |
| 4. Volatile dimethicone | 29.0 |
| 5. Trimethylsiloxysilicate | 20.0 |
| 6. Sorbitan sesquiisostearate | 1.5 |
| 7. Nonvolatile dimethicone | 1.3 |
| 8. Polyglyceryl triisostearate-2 | 2.0 |
| 9. Spherical Powder B-1 | 3.0 |
| 10. Synthetic mica base pearl agent | 7.0 |
| (particle diameter: 95 μm) | |
| 11. Synthetic mica base pearl agent | 3.0 |
| (particle diameter: 60 μm) | |
| 12. Synthetic mica base pearl agent | 10.0 |
| (particle diameter: 30 μm) | |
| 13. Synthetic mica | 4.18 |
| 14. Titanium oxide | 5.0 |
| 15. Blue 1 | 1.8 |
| 16. Yellow 4 | 0.1 |
| 17. Antioxidant | 0.02 |

### <Production Method>

The powder component was mixed with a mixture in which the oily component was dissolved at 80 to 100° C., and the mixture was uniformly dispersed using a 3 roll to obtain a cosmetic base material. The obtained cosmetic base material was filled in a mold and molded into a cylindrical shape having a diameter of 3 mm.

### <Evaluation>

The obtained eyeliner was evaluated in the same manner as described above, and consequently it was confirmed that the eyeliner was evaluated as creamy feel when used “◯”, clarity of contour of drawn lines “⊙”, no distortion “⊙”, matte finish “⊙”, good color development (concealing property) “◯”, secondary adhesion resistance “⊙”, and moldability “⊙”. The above eyeliner was evaluated for pearlescent finish in addition to matte finish, and the result was evaluated as “⊙”.

### Example 30: Eyeliner

| (Component) | (blending ratio (% by mass)) |
|---|---|
| 1. Rice bran wax | 2.5 |
| 2. Polyethylene | 8.5 |
| 3. Candelilla wax | 2.7 |
| 4. Volatile dimethicone | 26.0 |
| 5. Trimethylsiloxysilicate | 24.5 |
| 6. Sorbitan sesquiisostearate | 1.5 |
| 7. Nonvolatile dimethicone | 1.3 |
| 8. Diisostearyl malate | 2.0 |
| 9. Spherical Powder B-1 | 4.0 |
| 10. Hydrophobic synthetic mica | 0.38 |
| 11. Titanium oxide | 4.0 |
| 12. Ultramarine blue | 9.0 |
| 13. Black iron oxide | 4.0 |
| 14. Blue 1 | 7.5 |
| 15. Yellow 4 | 2.1 |
| 16. Antioxidant | 0.02 |

### <Production Method>

The powder component was mixed with a mixture in which the oily component was dissolved at 80 to 100° C., and the mixture was uniformly dispersed using a 3 roll to obtain a cosmetic base material. The obtained cosmetic base material was filled in a mold and molded into a cylindrical shape having a diameter of 2 mm.

### <Evaluation>

The obtained eyeliner was evaluated in the same manner as described above, and consequently it was confirmed that the eyeliner was evaluated as creamy feel when used “⊙”, clarity of contour of drawn lines “⊙”, no distortion “⊙”, matte finish “⊙”, good color development (concealing property) “◯”, secondary adhesion resistance “⊙”, and moldability “⊙”.

### Example 31: Concealer

| (Component) | (blending ratio (% by mass)) |
|---|---|
| 1. Rice bran wax | 1.5 |
| 2. Polyethylene | 2.0 |
| 3. Candelilla wax | 1.0 |
| 4. Volatile dimethicone | 20.0 |
| 5. Trimethylsiloxysilicate | 10.0 |
| 6. Sorbitan sesquiisostearate | 1.5 |
| 7. Nonvolatile dimethicone | 2.0 |
| 8. Diisostearyl malate | 3.0 |
| 9. Spherical Powder B-1 | 10.0 |
| 10. Yellow iron oxide | 5.0 |
| 11. Black iron oxide | 0.1 |
| 12. Red iron oxide | 1.5 |
| 13. Hydrophobic synthetic mica | 12.38 |
| 14. Titanium oxide | 30.0 |
| 15. Antioxidant | 0.02 |

### <Production Method>

The powder component was mixed with a mixture in which the oily component was dissolved at 80 to 100° C., and the mixture was uniformly dispersed using a 3 roll to obtain a cosmetic base material. The obtained cosmetic base material was filled in a mold and molded into a cylindrical shape having a diameter of 10 mm.

### <Evaluation>

The obtained concealer was evaluated in the same manner as described above except that it was applied to the back of the hand using the retractable stick type container, and consequently it was confirmed that it was evaluated as creamy feel when used “⊙”, clarity of contour of drawn lines “⊙”, no distortion “⊙”, matte finish “⊙”, good color development (concealing property) “⊙”, secondary adhesion resistance “⊙”, and moldability “⊙”.

### Example 32: Eyeliner

| (Component) | (blending ratio (% by mass)) |
|---|---|
| 1. Rice bran wax | 4.1 |
| 2. Polyethylene | 12.2 |
| 3. Candelilla wax | 3.7 |
| 4. Volatile dimethicone | 30.0 |
| 5. Trimethylsiloxysilicate | 25.0 |
| 6. Sorbitan sesquiisostearate | 2.0 |
| 7. Nonvolatile dimethicone | 1.3 |
| 8. Diisostearyl malate | 2.0 |
| 9. Spherical Powder B-1 | 1.0 |
| 10. Hydrophobic synthetic mica | 1.0 |
| 11. Black iron oxide | 1.6 |
| 12. Red iron oxide | 13.6 |
| 13. Titanium oxide | 2.5 |

### <Production Method>

The powder component was mixed with a mixture in which the oily component was dissolved at 80 to 100° C., and the mixture was uniformly dispersed using a 3 roll to obtain a cosmetic base material. The obtained cosmetic base material was filled in a mold and molded into a cylindrical shape having a diameter of 2 mm.

### <Evaluation>

The obtained eyeliner was evaluated in the same manner as described above, and consequently it was confirmed that the eyeliner was evaluated as creamy feel when used “◯”, clarity of contour of drawn lines “⊙”, no distortion “⊙”, matte finish “⊙”, good color development (concealing property), secondary adhesion resistance “⊙”, and moldability

### Example 33: Eyeliner

| (Component) | (blending ratio (% by mass)) |
|---|---|
| 1. Rice bran wax | 3.0 |
| 2. Polyethylene | 9.0 |
| 3. Candelilla wax | 2.7 |
| 4. Sorbitan sesquiisostearate | 1.5 |
| 5. Nonvolatile dimethicone | 1.3 |
| 6. Diisostearyl malate | 2.0 |
| 7. Isododecane | 26.0 |
| 8. Spherical Powder B-1 | 4.0 |
| 9. Hydrophobic synthetic mica | 0.97 |
| 10. Titanium oxide | 3.2 |
| 11. Yellow iron oxide | 0.01 |
| 12. Black iron oxide | 2.0 |
| 13. Red iron oxide | 19.8 |
| 14. Trimethylsiloxysilicate | 24.5 |
| 15. Antioxidant | 0.02 |

### <Production Method>

The powder component was mixed with a mixture in which the oily component was dissolved at 80 to 100° C., and the mixture was uniformly dispersed using a 3 roll to obtain a cosmetic base material. The obtained cosmetic base material was filled in a mold and molded into a cylindrical shape having a diameter of 2 mm.

### <Evaluation>

The obtained eyeliner was evaluated in the same manner as described above, and consequently it was confirmed that the eyeliner was evaluated as creamy feel when used “⊙”, clarity of contour of drawn lines “⊙”, no distortion “◯”, matte finish “⊙”, good color development (concealing property) “⊙”, secondary adhesion resistance “⊙”, and moldability “⊙”.

### Example 34: Eyeliner

| (Component) | (blending ratio (% by mass)) |
|---|---|
| 1. Rice bran wax | 2.3 |
| 2. Polyethylene | 8.0 |
| 3. Candelilla wax | 2.5 |
| 4. Nonvolatile dimethicone | 1.3 |
| 5. Diisostearyl malate | 3.5 |
| 6. Methyl trimethicone | 28.0 |
| 7. Spherical Powder B-1 | 3.0 |
| 8. Hydrophobic synthetic mica | 1.68 |
| 9. Yellow iron oxide | 3.0 |
| 10. Black iron oxide | 12.0 |
| 11. Red iron oxide | 10.2 |
| 12. Trimethylsiloxysilicate | 24.5 |
| 13. Antioxidant | 0.02 |

### <Production Method>

The powder component was mixed with a mixture in which the oily component was dissolved at 80 to 100° C., and the mixture was uniformly dispersed using a 3 roll to obtain a cosmetic base material. The obtained cosmetic base material was filled in a mold and molded into a cylindrical shape having a diameter of 2 mm.

### <Evaluation>

The obtained eyeliner was evaluated in the same manner as described above, and consequently it was confirmed that the eyeliner was evaluated as creamy feel when used “⊙”, clarity of contour of drawn lines “⊙”, no distortion “⊙”, matte finish “⊙”, good color development (concealing property) “⊙”, secondary adhesion resistance “⊙”, and moldability “⊙”.

What is claimed is:

1. An oily stick-shaped cosmetic comprising:
(A) a solid oil; (B) a spherical powder having an average particle diameter of 1 to 50 μm; (C) a volatile component; (D) a coating agent; and (E) a coloring material,
and containing (A1) rice bran wax as the (A) component.

2. The oily stick-shaped cosmetic according to claim 1, wherein a content of the (A) component is 3 to 25% by mass based on a total amount of the oily stick-shaped cosmetic.

3. The oily stick-shaped cosmetic according to claim 1, wherein a content of the (A1) component is 0.1 to 10% by mass based on a total amount of the oily stick-shaped cosmetic.

4. The oily stick-shaped cosmetic according to claim 1, wherein a content of the (A1) component is 1 to 70% by mass based on a total amount of the (A) component.

5. The oily stick-shaped cosmetic according to claim 1, further comprising
(A2) a hydrocarbon-based wax as the (A) component,
wherein a mass ratio [(A1)/(A2)] between the (A1) component and a hydrocarbon contained in (A2) the hydrocarbon-based wax is 1/30 to 2/1.

6. The oily stick-shaped cosmetic according to claim 1, wherein the (B) component is spherical silica including a metal oxide.

7. The oily stick-shaped cosmetic according to claim 1, wherein the (D) component is trimethylsiloxysilicate.

8. The oily stick-shaped cosmetic according to claim 1, wherein a content of the (C) component is 10 to 40% by mass based on a total amount of the oily stick-shaped cosmetic.

9. The oily stick-shaped cosmetic according to claim 1, wherein a total content of a pasty oil and a liquid oil contained in the oily stick-shaped cosmetic is 0.1 to 20% by mass based on a total amount of the oily stick-shaped cosmetic.

10. The oily stick-shaped cosmetic according to claim 1, wherein a ratio of a content of the (A) component to a total content of a pasty oil and a liquid oil contained in the oily stick-shaped cosmetic is 0.1 to 30.

11. An oily stick-shaped cosmetic comprising:
(A) a solid oil; (B) a spherical powder having an average particle diameter of 1 to 50 μm; (C) a volatile component; (D) a coating agent; and (E) a coloring material,
and containing (A1) rice bran wax as the (A) component,
wherein the (B) component is spherical silica including a metal oxide, and
the (D) component is trimethylsiloxysilicate.

12. The oily stick-shaped cosmetic according to claim 11, further comprising
(A2) a hydrocarbon-based wax as the (A) component,
wherein a mass ratio [(A1)/(A2)] between the (A1) component and a hydrocarbon contained in (A2) the hydrocarbon-based wax is 1/30 to 2/1.
